(19) United States
(12) Patent Application Publication
(10) Pub. No.: US 2010/0010049 A1
(43) Pub. Date: Jan. 14, 2010

(54) Novel Tyrosinase inhibitors and pharmaceutical/cosmetic applications thereof

(75) Inventors: Itaru Suzuki, Fayence (FR); Irène Erdelmeier, Paris (FR); Jean-Claude Yadan, Montreuil Sous Bois (FR); Isabelle Pelisson, Vallauris (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 12/318,902

(22) Filed: Jan. 12, 2009

Related U.S. Application Data
(63) Continuation of application No. PCT/EP2007/005019, filed on Jul. 10, 2007.
(60) Provisional application No. 60/831,210, filed on Jul. 17, 2006.

(30) Foreign Application Priority Data
Jul. 11, 2006 (FR) 0652908

(51) Int. Cl. A61K 31/4439; A61K 31/415; C07D 233/00; C07D 401/12

(52) U.S. Cl. 514/341; 514/392; 548/317.1; 546/274.4

(57) ABSTRACT

Compounds corresponding to the following general formula (I):

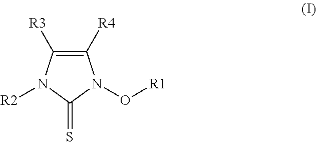

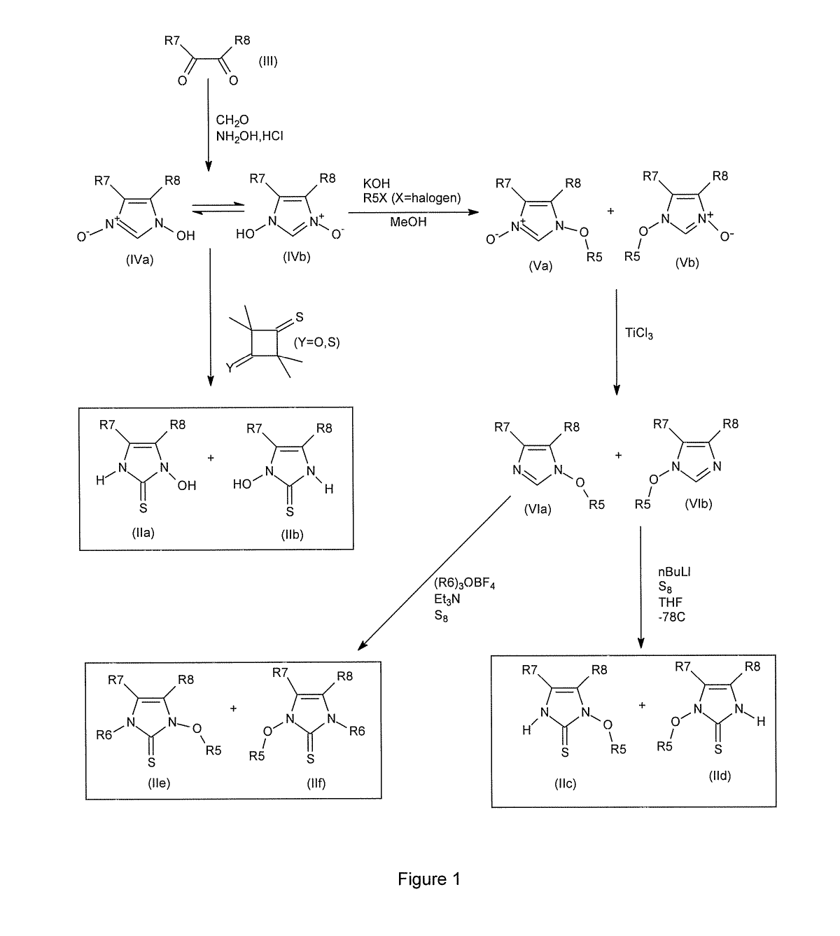

are formulated into pharmaceutical compositions useful in human or veterinary medicine or else into cosmetic compositions; novel compounds of formula (II) are also thus useful:

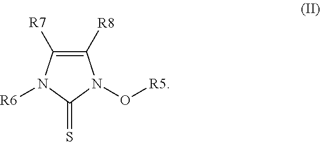

### CROSS-REFERENCE TO PROVISIONAL/PCT APPLICATIONS

This application claims priority under 35 U.S.C. §119 of FR 0652908, filed Jul. 11, 2006 and U.S. Provisional Application No. 60/831,210, filed Jul. 17, 2006, and is a continuation/national phase of PCT/EP 2007/057019, filed Jul. 10, 2007 and designating the United States (published in the English language on Jan. 17, 2008 as WO 2008/006824 A1); each hereby expressly incorporated by reference in its entirety and each assigned to the assignee hereof.

### BACKGROUND OF THE INVENTION

1. Technical Field of the Invention

The present invention relates to the novel administration of compounds which are tyrosinase inhibitors in the treatment of hyperpigmentary disorders.

The present invention also relates, as novel and useful industrial products, to novel compounds which are tyrosinase inhibitors. It also relates to their process of preparation and to their formulation into pharmaceutical compositions useful in human or veterinary medicine or else into cosmetic compositions and to the non-therapeutic applications thereof.

2. Description of Background and/or Related and/or Prior Art

The pigmentation of the human skin results from the synthesis of melanin by the dendritic cells, the melanocytes. The latter comprise organells, referred to as melanosomes, which are the site of the biosynthesis of melanin. These are the melanosomes which, after migration along the dendrites, are transferred from the melanocytes to the keratinocytes. The keratinocytes are then transported to the surface of the skin during the process of differentiation of the epidermis (Gilchrest B A, Park H Y, Eller M S, Yaar M, Mechanisms of ultraviolet light-induced pigmentation, Photochem. Photobiol., 1996, 63, 1-10; Hearing V J, Tsukamoto K, Enzymatic control of pigmentation in mammals, FASEB J., 1991, 5, 2902-2909).

Among the enzymes of melanogenesis, tyrosinase is a key enzyme which catalyses the first two stages of the synthesis of melanin. Homozygous tyrosinase mutations result in oculocutaneous albinism type I characterized by a complete absence of the synthesis of melanin (Toyofuku K, Wada I, Spritz R A, Hearing V J, The molecular basis of oculocutaneous albinism type 1 (OCA1): sorting failure and degradation of mutant tyrosinases results in a lack of pigmentation. Biochem. J., 2001, 355, 259-269).

Given that hyperpigmentation disorders result from an increase in melanin production, it proves to be important to develop novel therapeutic approaches, the rational of which is based on the inhibition of the activity of tyrosinase.

The majority of skin-lightening compounds already known are phenols/catechols. These compounds inhibit tyrosinase but most of these compounds are cytotoxic towards melanocytes, which might bring about permanent depigmentation of the skin.

It thus appears advantageous, for application to man, to have available novel compounds which are tyrosinase inhibitors having both good effectiveness and good tolerance.

Compounds derived from 3,5-diphenyl-1-hydroxy-1,2-dihydroimidazole-2-thiones are described in U.S. Pat. No. 5,283,271 as anti-microbials.

Hauser describes a synthesis of 1-benzyloxy-2,3-dihydroimidazole-2-thione and the preparation of 1-benzyloxy-1H-imidazoles (H. Hauser et al., Sci. Pharma., 56, 235-241, 1988).

### SUMMARY OF THE INVENTION

Certain imidazolethione compounds have now been discovered that have an inhibitory effect on the enzyme tyrosinase. These compounds have applications in human medicine, in particular in dermatology, and in the cosmetics field.

Thus, the present invention features formulation of the compounds of following formula (I):

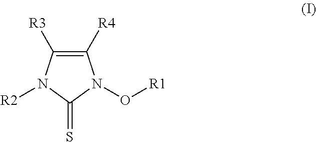

in which:
R1 is a hydrogen atom, an alkyl radical, an aralkyl radical or a cycloalkyl radical,
R2 is a hydrogen atom, an alkyl radical, an aryl radical or an aralkyl radical,
R3 is a hydrogen atom, an alkyl radical, an aryl radical or an aralkyl radical,
R4 is a hydrogen atom, an alkyl radical, an aryl radical or an aralkyl radical,
   and the salts of the compounds of formula (I) and also their optical, tautomeric and geometric isomers, into pharmaceutical compositions useful for the treatment and/or prevention of hyperpigmentary disorders.

According to the present invention, the preferred compounds of formula (I) are those for which at least one and preferably all of the following conditions are observed:
R1 is a hydrogen atom, an alkyl radical or an aralkyl radical,
R2 is a hydrogen atom, an alkyl radical or an aryl radical,
R3 is a hydrogen atom, an alkyl radical or an aryl radical,
R4 is a hydrogen atom, an alkyl radical or an aryl radical,
   and the salts of the said preferred compounds of formula (I) and also their optical, tautomeric and geometric isomers.

According to the present invention, the particularly preferred compounds of formula (I) are those for which at least one and preferably all of the following conditions are observed:
R1 is a hydrogen atom,
R2 is a hydrogen atom,
R3 is a hydrogen atom or an aryl radical,
R4 is a hydrogen atom or an aryl radical,
   and the salts of the said particularly preferred compounds of formula (I) and also their optical, tautomeric and geometric isomers.

Exemplary compounds of formula (I) within the scope of the present invention include the following:
a. 1-benzyloxy-1,3-dihydroimidazole-2-thione
b. 3-hydroxy-1,4-diphenyl-1,3-dihydroimidazole-2-thione
c. 3-hydroxy-1-(4-methoxyphenyl)-4-phenyl-1,3-dihydroimidazole-2-thione
d. 1-hydroxy-4-methyl-3,5-diphenyl-1,3-dihydroimidazole-2-thione
e. 1-benzyloxy-3-methyl-1,3-dihydroimidazole-2-thione

Furthermore, it has also now been discovered, surprisingly and unexpectedly, that certain novel compounds have an inhibitory activity with regard to tyrosinase and a low toxicity.

Thus, the present invention also features novel compounds corresponding to the following general formula (II):

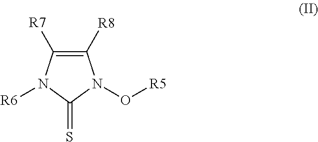

in which:
R5 is a hydrogen atom, an alkyl radical, an aralkyl radical or a cycloalkyl radical,
R6 is a hydrogen atom, an alkyl radical, an aryl radical or an aralkyl radical,
R7 is a hydrogen atom, an alkyl radical, an aryl radical or an aralkyl radical,
R8 is a hydrogen atom, an alkyl radical, an aryl radical or an aralkyl radical,
   with the provisos that:
when R5 is a hydrogen atom, then R6 cannot represent a substituted or unsubstituted phenyl radical;
when R6 is a hydrogen atom or a methyl and R7 and R8 are each a hydrogen atom, then R5 cannot represent an unsubstituted benzyl radical;
when R5 and R6 are each a hydrogen atom and R7 is a phenyl radical, then R8 cannot represent a methyl,
   and the salts of the compounds of formula (II) and also their optical, tautomeric and geometric isomers. Preferably, when R5 and R6 are each a hydrogen atom and R7 is a methyl radical, then R8 cannot represent an unsubstituted phenyl radical. In other words, preferably, the compound 1-hydroxy-4-methyl-5-phenyl-1,3-dihydroimidazole-2-thione is excluded from the compounds of formula (II) according to the invention.

When the compounds according to the invention are provided in the form of a salt, it is preferably a salt of an alkali metal or alkaline earth metal or also a salt of zinc or of an organic amine.

The term “optical isomers” means enantiomers or diastereoisomers, the first being characterized in that two molecular entities are images of one another in a mirror and are not superimposable and the second being characterized in that they belong to a group of stereoisomers which are not enantiomers. The term “stereoisomers” means isomers which differ from one another only in the arrangement of their atoms in space.

The term “tautomeric isomers” means isomers, the structures of which differ in the position of an atom, generally a hydrogen atom, and of one or more multiple bonds, and which are capable of easily and reversibly changing from one to another.

The term “geometric isomers” means a cis/trans or E/Z isomerism. More particularly, the possible double bond(s) present in the various substituents can be of E or Z configuration.

According to the present invention, the term “alkyl radical” is a saturated, linear or branched, hydrocarbon chain having from 1 to 6 carbon atoms, such as, for example, the methyl, ethyl, n-propyl, isopropyl, n-butyl, isobutyl, tert-butyl, n-pentyl or n-hexyl radicals.

The term “aryl radical” means a phenyl radical, a biphenyl radical or a naphthyl radical or also a heteroaromatic radical, such as, for example, a thiophenyl, a pyridinyl, a pyrimidinyl, an imidazolyl or a triazolyl, it being possible for the said aryl radical optionally to be fused with one or more other rings and it being possible for the said aryl radical optionally to be mono- or disubstituted by one or more substituents selected from a halogen atom, a CF₃ radical, an alkyl radical, an alkoxy radical, a nitro functional group, an alkyl ester group, a nitrile functional group, an amide functional group, a carboxyl functional group, a hydroxyl radical and an amino functional group optionally substituted by at least one alkyl radical.

Preferably, the aryl radical is selected from the unsubstituted phenyl radical, the phenyl radical substituted by at least one alkoxy radical (preferably methoxy radical), the thiophenyl radical, the pyridinyl radical substituted by at least one alkoxy radical (preferably methoxy radical) and the phenyl radical substituted by at least one nitro functional group, preferably two nitro functional groups.

The term “aralkyl radical” is a —(CH₂)ₙ-aryl radical with n ranging from 1 to 6 inclusive and aryl is as defined above.

Preferably, the aralkyl radical is selected from the unsubstituted benzyl radical and the benzyl radical substituted by at least one halogen atom (preferably fluorine).

The term “alkoxy radical” is an oxygen atom substituted by an alkyl radical as defined above.

Preferably, the alkoxy radical is selected from the methoxy, ethoxy, n-propoxy, isopropoxy, n-butoxy, isobutoxy, tert-butoxy, n-pentyloxy and n-hexyloxy radicals.

The term “cycloalkyl radical” is a saturated cyclic hydrocarbon chain comprising from 3 to 8 carbon atoms.

The term “halogen atom” means preferably a chlorine, fluorine, bromine or iodine atom.

According to the present invention, the preferred compounds of formula (II) are those for which at least one and preferably all of the following conditions are observed:
R5 is a hydrogen atom, an alkyl radical or an aralkyl radical,
R6 is a hydrogen atom, an alkyl radical or an aryl radical,
R7 is a hydrogen atom, an alkyl radical or an aryl radical,
R8 is a hydrogen atom, an alkyl radical or an aryl radical,
   and the salts of the said preferred compounds of formula (II) and also of their optical, tautomeric and geometric isomers.

According to the present invention, the particularly preferred compounds of formula (II) are those for which at least one and preferably all of the following conditions are observed:
R5 is a hydrogen atom,
R6 is a hydrogen atom,
R7 is a hydrogen atom, an alkyl radical or an aryl radical,
R8 is a hydrogen atom or an aryl radical,
   and the salts of the said particularly preferred compounds of formula (II) and also of their optical, tautomeric and geometric isomers.

Exemplary compounds of formula (II) within the scope of the present invention include the following:
1. 1-methoxy-1,3-dihydroimidazole-2-thione
2. 1-(pyridin-3-ylmethoxy)-1,3-dihydroimidazole-2-thione
3. 1-methoxy-3-methyl-1,3-dihydroimidazole-2-thione
4. 1-hydroxy-5-phenyl-1,3-dihydroimidazole-2-thione
5. 1-hydroxy-4-phenyl-1,3-dihydroimidazole-2-thione
6. 1-hydroxy-5-(4-methoxyphenyl)-1,3-dihydroimidazole-2-thione
7. 1-hydroxy-4-(4-methoxyphenyl)-1,3-dihydroimidazole-2-thione
8. 1-hydroxy-5-(thiophen-2-yl)-1,3-dihydroimidazole-2-thione
9. 1-benzyloxy-4-phenyl-1,3-dihydroimidazole-2-thione
10. 1-benzyloxy-5-phenyl-1,3-dihydroimidazole-2-thione
11. 1-ethoxy-4-phenyl-1,3-dihydroimidazole-2-thione
12. 1-ethoxy-5-phenyl-1,3-dihydroimidazole-2-thione
13. 1-ethoxy-4-methyl-1,3-dihydroimidazole-2-thione
14. 1-ethoxy-5-methyl-1,3-dihydroimidazole-2-thione
15. 1-ethyl-3-methoxy-1,3-dihydroimidazole-2-thione
16. 1-benzyloxy-3-ethyl-1,3-dihydroimidazole-2-thione
17. 1-(3,5-difluorobenzyloxy)-1,3-dihydroimidazole-2-thione
18. 1-hydroxy-4-(3-nitrophenyl)-1,3-dihydroimidazole-2-thione
19. 1-hydroxy-5-(3-nitrophenyl)-1,3-dihydroimidazole-2-thione
20. 1-hydroxy-4-(4-nitrophenyl)-1,3-dihydroimidazole-2-thione
21. 1-hydroxy-5-(4-nitrophenyl)-1,3-dihydroimidazole-2-thione
22. 1-hydroxy-3-methyl-5-phenyl-1,3-dihydroimidazole-2-thione
23. 1-hydroxy-3-methyl-4-phenyl-1,3-dihydroimidazole-2-thione
24. 1-methoxy-4-phenyl-1,3-dihydroimidazole-2-thione
25. 1-methoxy-5-phenyl-1,3-dihydroimidazole-2-thione

The present invention also features processes for the preparation of the compounds of general formula (II).

### BRIEF DESCRIPTION OF THE DRAWING

The FIGURE of Drawing illustrates the main stages of the reaction scheme for the preparation of the compounds of the invention.

### DETAILED DESCRIPTION OF BEST MODE AND SPECIFIC/PREFERRED EMBODIMENTS OF THE INVENTION

Specifically, the compounds of general formula (II) in which R5 and R6 are each a hydrogen atom and R7 and R8, which are identical or different, are each a hydrogen atom, an alkyl radical, an aryl radical or an aralkyl radical are prepared in the following manner:
a) addition of an aqueous formaldehyde solution to a compound of general formula (III):

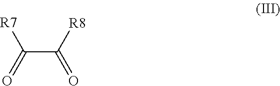

in solution in a polar solvent;

The compounds of general formula (III) are either commercially available or are obtained according to processes described in the literature.
b) addition of an aqueous hydroxylamine hydrochloride solution to the mixture obtained in a), at a temperature preferably of from −30° C. to 15° C.;
c) addition of a hydrochloric acid solution to the mixture obtained in b), at a temperature preferably of from −30° C. to 15° C.

After having allowed the reaction medium to return to ambient temperature, it is stirred, preferably for from 2 h to 72 h.
d) after neutralizing and obtaining a precipitate, the following stage is carried out:
e) Filtering and drying the precipitate obtained in d), corresponding to the compounds of general formulae (IVa) and/or (IVb):

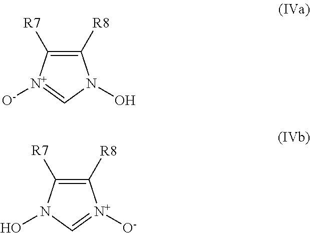

f) treatment of the compounds obtained in e) with a sulfur donor in order to obtain the compounds of general formula (II).

The compounds obtained in e) are treated at a temperature preferably of from −30° C. to 15° C. with a sulfur donor, such as, for example, 2,2,4,4-tetramethyl-1,3-cyclobutadithione or 2,2,4,4-tetramethyl-3-thioxocyclobutanone. After stirring for a few hours, the reaction medium is filtered. After washing and evaporating the filtrate, the precipitate obtained corresponds to the compounds of general formula (IIa) and/or (IIb), which are compounds of general formula (II):

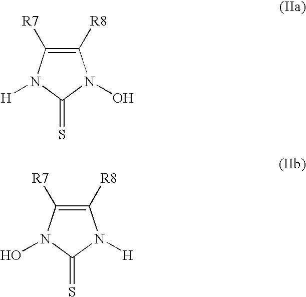

Each compound (IIa) and (IIb) is isolated separately after purification, for example by liquid chromatography on silica gel or by recrystallization.

Specifically, the compounds of general formula (II) in which R5 is an alkyl radical, an aralkyl radical or a cycloalkyl radical, R6 is a hydrogen atom and R7 and R8, which are identical or different, are each a hydrogen atom, an alkyl radical, an aryl radical or an aralkyl radical are prepared in the following manner:

Stages a) to e) are identical to the stages described above;
f) alkylation of the compounds obtained in e) in order to obtain the compounds of general formula (Va) and/or (Vb):

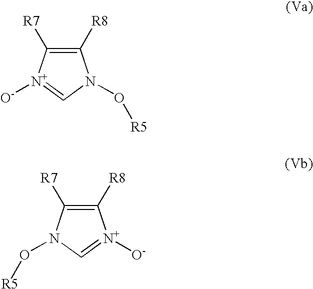

Preferably, the alkylation of the compounds obtained in e) is carried out in the presence of a base and of an alkylating agent.
g) reduction of the compounds of general formula (Va) and/or (Vb) in order to obtain the compounds of general formula (VIa) and/or (VIb).

Specifically, the compounds of general formula (Va) and/or (Vb) are reduced using a reducing agent, such as titanium trichloride, in an organic solvent, preferably at a temperature of from −30° C. to 15° C. and in the presence of an inorganic acid, such as hydrochloric acid. The product obtained corresponds to the compounds of general formula (VIa) and/or (VIb).

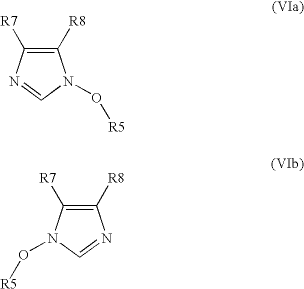

h) treatment of the compounds of general formulae (VIa) and/or (VIb) with n-butyllithium in an organic solvent and then with sulfur S₈, in order to obtain the compounds of general formula (II).

Specifically, the compounds of general formulae (VIa) and/or (VIb) are treated with n-butyllithium in an organic solvent, such as, for example, tetrahydrofuran, at a temperature preferably of from −80° C. to −30° C., and then they are reacted with sulfur S₈. After treatment, the product obtained corresponds to the compounds of general formula (IIc) and/or (IId), which are compounds of general formula (II):

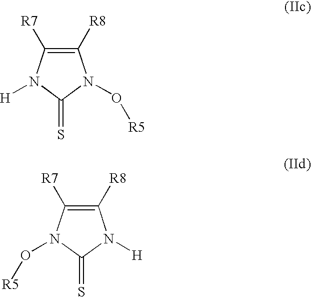

Each compound (IIc) and (IId) is isolated separately after purification, for example by liquid chromatography on silica gel or by recrystallization.

Specifically, the compounds of general formula (II) in which R5 is an alkyl radical, an aralkyl radical or a cycloalkyl radical, R6 is an alkyl radical or an aralkyl radical and R7 and R8, which are identical or different, are each a hydrogen atom, an alkyl radical, an aryl radical or an aralkyl radical are prepared in the following manner:

Stages a) to g) are identical to the stages described above:
h) alkylation of the compounds obtained in g):

Preferably, the compounds obtained in stage g), corresponding to the compounds of general formulae (VIa) and/or (VIb):

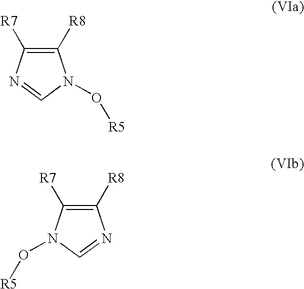

are alkylated in an organic solvent of dichloromethane type in the presence of an alkylating agent;
i) Addition of a base and of sulfur S₈ to the reaction medium obtained in h), in order to obtain the compounds of general formula (II).

Specifically, a base of triethylamine type and sulfur S₈ are added to the reaction medium, which is stirred for a few hours. After treatment, the product obtained corresponds to the compounds of general formulae (IIe) and (IIf), which are compounds of general formula (II):

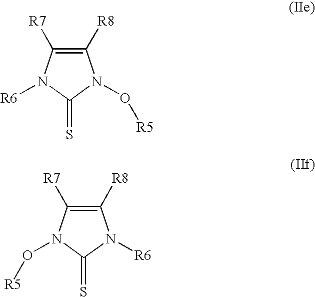

Each compound (IIe) and (IIf) is isolated separately after purification, for example by liquid chromatography on silica gel or by recrystallization.

The compounds (IIa), (IIb), (IIc), (IId), (IIe) and (IIf) are compounds of general formula (II).

The term “alkylating agent” means in particular trialkyloxonium derivatives, alkyl sulfates and alkyl or aralkyl halides or triflates.

The compounds according to the invention exhibit inhibitory properties with regard to tyrosinase. This activity is measured in an enzymatic assay by the inhibition constant IC₅₀ (dose which inhibits 50% of the enzymatic activity).

The term “tyrosinase inhibitors” means, according to the invention, any compound exhibiting an inhibition constant with regard to the enzymes tyrosine hydroxylase and DOPA oxidase of less than or equal to 500 μM in an enzymatic assay as described in Example 9.

The preferred compounds of the present invention exhibit an inhibition constant of less than or equal to 100 μM and advantageously of less than or equal to 10 μM, for example of less than or equal to 1 μM.

The present invention also features administration of the compounds of formula (II) as described above as medicaments.

This invention also features formulation of the compounds of formula (I) into pharmaceutical compositions useful for the treatment or prevention of hyperpigmentary disorders.

The compounds according to the invention are particularly well suited for the treatment or prevention of hyperpigmentary disorders, such as melasma, chloasma, lentigines, senile lentigo, vitiligo, freckles, post-inflammatory hyperpigmentations due to an abrasion and/or a burn and/or a scar and/or a dermatosis and/or a contact allergy; nevi, genetically determined hyperpigmentations, hyperpigmentations of metabolic or drug origin, melanomas or any other hyperpigmentary lesion.

The present invention also features novel medicinal compositions useful in particular for the treatment of the abovementioned conditions and which are characterized in that they comprise, in a physiologically acceptable vehicle compatible with the method of administration selected for the composition, at least one compound of formula (I), one of its isomers or one of its salts.

Specifically, this invention features in particular pharmaceutical compositions comprising, in a physiologically acceptable medium, at least one compound of formula (II) as defined above.

The term “physiologically acceptable medium” means a medium compatible with the skin, mucous membranes and/or superficial body growths.

The compositions according to the invention can be administered orally, enterally, parenterally, topically or ocularly. Preferably, the pharmaceutical composition is packaged in a form suitable for topical application.

The composition can be provided orally in the form of tablets, hard gelatine capsules, sugar-coated tablets, syrups, suspensions, solutions, powders, granules, emulsions, suspensions of microspheres or nanospheres or of lipid or polymeric vesicles which make possible controlled release. The composition can be provided parenterally in the form of solutions or suspensions for infusion or for injection.

The compounds according to the invention are generally administered at a daily dose of approximately 0.01 mg/kg to 100 mg/kg of body weight, taken 1 to 3 times.

The compounds are administered systemically at a concentration generally from 0.001 % to 10% by weight, preferably from 0.01 % to 1 % by weight, with respect to the weight of the composition.

The pharmaceutical compositions according to the invention are topically more particularly useful for the treatment of the skin and mucous membranes and can be provided in the liquid, pasty or solid form and more particularly in the form of ointments, creams, milks, salves, powders, impregnated pads, syndets, solutions, gels, sprays, foams, suspensions, sticks, shampoos or washing bases. Same can also be provided in the form of suspensions of microspheres or nanospheres or of lipid or polymeric vesicles or of polymeric or gelled patches which make possible controlled release.

The compounds are administered topically at a concentration generally of from 0.001% to 10% by weight, preferably from 0.01% to 1% by weight, with respect to the total weight of the composition.

The compounds of formula (I) according to the invention also find application in the cosmetics field, in particular for protecting against the harmful aspects of the sun, for preventing and/or combating photoinduced or chronological aging of the skin and superficial body growths.

This invention thus features in particular compositions comprising, in a cosmetically acceptable vehicle, at least one compound of formula (I), in particular at least one compound of formula (II).

The term “cosmetically acceptable medium” means a medium compatible with the skin, mucous membranes and/or superficial body growths.

The present invention also features the cosmetic application of a composition comprising at least one compound of formula (I), in particular at least one compound of formula (II), for preventing and/or treating signs of aging and/or dry skin.

This invention also features the cosmetic application of a composition comprising at least one compound of formula (I), in particular at least one compound of formula (II), for body or hair hygiene.

The cosmetic compositions according to the invention comprising, in a cosmetically acceptable vehicle, at least one compound of formula (I), in particular at least one compound of formula (II), or one of its optical or geometic isomers or one of its salts can be provided in particular in the form of a cream, a milk, a gel, suspensions of microspheres or nanospheres or lipid or polymeric vesicles, impregnated pads, solutions, sprays, foams, sticks, soaps, washing bases or shampoos.

The concentration of compound of formula (I), in particular a compound of formula (II), in the cosmetic composition preferably ranges from 0.001% to 3% by weight, with respect to the total weight of the composition.

The pharmaceutical and cosmetic compositions as described above can additionally comprise inert additives or even pharmacodynamically active additives, as regards the pharmaceutical compositions, or combinations of these additives, and in particular:
wetting agents;
flavor enhancers;
preservatives, such as para-hydroxybenzoic acid esters;
stabilizers;
moisture regulators;
pH-regulating agents;
osmotic pressure modifiers;
emulsifying agents;
UV-A and UV-B screening agents;
antioxidants, such as α-tocopherol, butylated hydroxyanisole or butylated hydroxytoluene, superoxide dismutase or ubiquinol;
emollients;
moisturizing agents, such as glycerol, PEG 400, thiamorpholinone and its derivatives, or urea.

Of course, one skilled in this art would take care to select the optional compound or compounds to be added to these compositions such that the advantageous properties intrinsically attached to the present invention are not, or not substantially, detrimentally affected by the envisaged addition.

In order to further illustrate the present invention and the advantages thereof, the following specific examples of active compounds of formula (II) according to the invention, biological activity results and various specific formulations based on such compounds are given, it being understood that same are intended only as illustrative and in nowise limitative. In said examples to follow, all parts and percentages are given by weight, unless otherwise indicated.

### Example 1

### Synthesis of 1-Methoxy-1,3-dihydroimidazole-2-thione

### a) Preparation of 3-hydroxyimidazole N-oxide

The compound 3-hydroxyimidazole N-oxide is prepared in one stage from glyoxal, formaldehyde and hydroxylamine hydrochloride according to the process described in B. L. Eriksen et al., J. Org. Chem., 63, 12-16 (1998). It is obtained in the form of a white solid. This product is used as is in the following stage.

Melting point=184°-186° C.

### b) Preparation of 3-methoxyimidazole 1-oxide

10 g (100 mmol) of the compound 3-hydroxyimidazole N-oxide are suspended in 100 ml of methanol. This mixture is placed in a cold bath (0° C.) and then 8.58 g (130 mmol) of 85% potassium hydroxide KOH are slowly added (T<20° C.). The mixture becomes homogeneous and 6.54 ml (14.9 g, 105 mmol) of iodomethane are added dropwise over 20 min. After heating under reflux for 22 h, 0.62 ml (10 mmol) of iodomethane is added and the mixture is stirred at ambient temperature overnight. The methanol is evaporated under vacuum and the mixture taken up in 100 ml of dichloromethane. After filtering off the potassium iodide, drying the filtrate over magnesium sulfate and evaporating, 6.93 g (61%) of 3-methoxyimidazole 1-oxide are obtained in the form of an oil.

### c) Preparation of 1-methoxyimidazole

6.64 g (58.2 mmol) of 3-methoxyimidazole 1-oxide are dissolved in 50 ml of methanol. This mixture is placed in a cold bath (0° C.) and 100 ml (128 mmol) of a 15% solution of titanium trichloride in 7N hydrochloric acid are added slowly over 25 min. The reaction mixture is allowed to return to ambient temperature and is then stirred for 2.5 h. After partial evaporation of the methanol, the mixture is poured into 500 ml of water and basified to pH=10. After extracting with 4×150 ml of ethyl acetate, drying over magnesium sulfate and evaporating the solvents, 2.28 g (40%) of 1-methoxyimidazole are obtained in the form of an orange oil.

### d) Synthesis of 1-methoxy-1,3-dihydroimidazole-2-thione

2.28 g (23.2 mmol) of 1-methoxyimidazole are dissolved under argon in 186 ml of anhydrous THF. The reaction mixture is cooled to −78° C. and then 14.52 ml (23.3 mmol) of a 1.6M solution of n-butyllithium in n-hexane are added dropwise. Ten minutes after the end of the addition, 1.49 g (46.4 mmol) of sulfur are added and the cold bath is removed. After stirring at ambient temperature for 1.5 h, the mixture is poured onto 250 ml of a saturated ammonium chloride solution. After extracting with dichloromethane (2×250 ml and 1×120 ml), drying over magnesium sulfate, filtering and evaporating the solvents, 3.01 g of a red amorphous solid are obtained. After recrystallizing from a cyclohexane/ethyl acetate mixture (150 ml, 50/50), 950 mg (30%) of the compound 1-methoxy-1,3-dihydroimidazole-2-thione are obtained in the form of colorless crystals.

Melting point=111.0°-111.8° C.

¹H NMR (CDCl₃, 200 MHz): δ (ppm)=4.12 (s, 3H, —OCH₃); 6.65 (d, J=3 Hz, 1H); 6.91 (d, J=3 Hz, 1H).

¹³C NMR (CDCl₃, 50 MHz): δ (ppm)=65.3; 111.8; 114.7; 156.1.

MS (Cl, NH₃): m/z=148 (M+NH₄)⁺; 131 (MH)⁺.

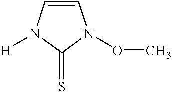

### Example 2

### Synthesis of 1-(Pyridin-3-ylmethoxy)-1,3-dihydroimidazole-2-thione

### a) Preparation of 3-(pyridin-3-ylmethoxy)imidazole 1-oxide

1.98 g (19.8 mmol) of the compound 3-hydroxyimidazole N-oxide obtained in Example 1a) are suspended in 20 ml of methanol. This mixture is placed in a cold bath (0° C.) and then 3.01 g (45.6 mmol) of 85% potassium hydroxide KOH are slowly added (T<20° C.). The mixture becomes homogeneous and 5.17 g (19.8 mmol) of 3-bromomethylpyridine hydrobromide are added. After heating under reflux for 22 h, the methanol is evaporated under vacuum and the mixture taken up in 50 ml of dichloromethane. After filtering off the potassium bromide, drying the filtrate over magnesium sulfate and evaporating the solvents, 3.65 g of 3-(pyridin-3-ylmethoxy)imidazole 1-oxide are obtained and used as is in the following stage.

### b) Preparation of 1-(pyridin-3-ylmethoxy)imidazole

3.64 g (19.8 mmol) of 3-(pyridin-3-ylmethoxy)imidazole 1-oxide are dissolved in 119 ml of methanol. This mixture is placed in a cold bath (0° C.) and 34 ml of a 15% solution of titanium trichloride in 7N hydrochloric acid are slowly added. The reaction mixture is allowed to return to ambient temperature and the mixture is then stirred for 48 h. After partial evaporation of the methanol, the mixture is poured into 50 ml of water and basified to pH=10. After extracting with 4×30 ml of ethyl acetate, drying over magnesium sulfate and evaporating the solvents, 2.58 g (74%) of 1-(pyridin-3-ylmethoxy)imidazole are obtained in the form of an orange oil.

### c) Synthesis of 1-(pyridin-3-ylmethoxy)-1,3-dihydroimidazole-2-thione:

4.86 g (27.7 mmol) of 1-(pyridin-3-ylmethoxy)imidazole are dissolved under argon in 55 ml of anhydrous THF. The reaction mixture is cooled to −78° C. and then 19.8 ml (27.7 mmol) of a 1.4M solution of n-butyllithium in n-hexane are added dropwise. Ten minutes after the end of the addition, 1.77 g (55.4 mmol) of sulfur are added and the cold bath is removed. The reaction mixture becomes gradually brown and remains heterogeneous. After stirring at ambient temperature for 30 min, the mixture is poured onto 60 ml of a saturated ammonium chloride solution. After extracting with dichloromethane (6×40 ml) and with ethyl acetate (2×40 ml), drying over magnesium sulfate, filtering and evaporating the solvents, 5.80 g of a brown amorphous solid are obtained. After chromatographing on silica gel (ethyl acetate/methanol, 90/10) and then crystallizing from a cyclohexane/ethyl acetate mixture (50/50,15 ml), 1.56 g (27%) of 1-(pyridin-3-ylmethoxy)-1,3-dihydroimidazole-2-thione are obtained in the form of a beige solid.

Melting point=128°-129° C.

¹H NMR (d₆-DMSO, 300 MHz): δ (ppm)=5.34 (s, 2H, —OCH₂Pyr); 6.83 (t, J=2.5 Hz, 1H); 7.22 (t, J=2.5 Hz, 1H); 7.46 (dd, J=7 Hz, J=4.8 Hz, 1H); 7.97 (m, 1 H); 8.61 (dd, J=4.8 Hz, J=1.5 Hz, 1 H); 8.69 (d, J=1.5 Hz, 1 H).

¹³C NMR (d₆-DMSO, 75.5 MHz): δ (ppm)=75.4; 111.7; 115.9; 123.6; 129.5; 137.5; 150.2; 150.5; 156.7.

MS (Cl, NH₃): m/z=225 (M+NH₄)⁺; 208 (M+H)⁺; 125; 108; 101.

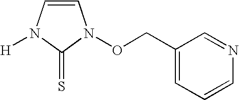

### Example 3

### Synthesis of 1-Methoxy-3-methyl-1,3-dihydroimidazole-2-thione

3.2 g (32.6 mmol) of 1-methoxyimidazole prepared in Example 1c) are dissolved under argon in 40 ml of anhydrous dichloromethane.

4.92 g (32.6 mmol) of trimethyloxonium tetrafluoroborate are added portionwise at ambient temperature. After stirring at ambient temperature for 1 h 30, 10 ml (7.26 g, 71.7 mmol) of triethylamine are added dropwise, followed by 2.31 g (71.7 mmol) of sulfur. After stirring at ambient temperature for 18 h, the mixture is poured onto 50 ml of methanol. The sulfur is filtered off and the solvents are evaporated. After recrystallizing the product obtained (1.92 g) from 50 ml of a cyclohexane/ethyl acetate mixture (50/50), 1.17 g (24%) of 1-methoxy-3-methyl-1,3-dihydroimidazole-2-thione are obtained in the form of a white solid.

Melting point=56.9°-57.5° C.

¹H NMR (CDCl₃, 200 MHz): δ (ppm)=3.56 (s, 3H, —NCH₃); 4.08 (s, 3H, —OCH₃); 6.58 (d, J=3 Hz, 1H); 6.88 (d, J=3 Hz, 1H)

¹³C NMR (CDCl₃, 50 MHz): δ (ppm)=34.9; 64.9; 113.3; 114.7; 158.2 MS (EI 70 eV): m/z=144 (M⁺); 114; 81; 72; 42.

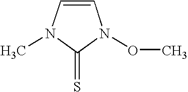

### Example 4

### Synthesis of 1-Hydroxy-5-phenyl-1,3-dihydroimidazole-2-thione

### a) Preparation of 3-Hydroxy-4(5)-phenylimidazole N-oxide

15 g (100 mmol) of phenylglyoxal monohydrate and 9 ml (120 mmol) of a 37% aqueous formaldehyde solution are dissolved in 22.5 ml of methanol.

17 ml of an aqueous solution comprising 13.9 g (200 mmol) of hydroxylamine hydrochloride are added dropwise at 0° C. At the end of the addition, the appearance of a white solid is observed.

2 ml of concentrated hydrochloric acid are subsequently added dropwise and then the reaction mixture is allowed to return to ambient temperature. After stirring for 4 days, the medium is cooled to 5° C. and 20 ml of concentrated sodium hydroxide solution are added. After stirring at this temperature for 1 h 30, the precipitate formed is filtered off and rinsed with 50 ml of cold water and then with 25 ml of cold methanol and 50 ml of ethyl ether. After drying under vacuum, 16.2 g (92%) of a 3-hydroxy-4(5)-phenylimidazole N-oxide mixture are obtained in the form of a white powder.

Melting point=211.6°-212.7° C.

### b) Synthesis of 1-Hydroxy-5-phenyl-1,3-dihydroimidazole-2-thione

100 g (0.57 mmol) of 3-hydroxy-4(5)-phenylimidazole N-oxide are suspended in 2 liters of dichloromethane. A solution of 240 g (1.40 mmol) of 2,2,4,4-tetramethyl-3-thioxocyclobutanone in 500 ml of dichloromethane is added under cold conditions (5° C.) and the reaction mixture is allowed to return to ambient temperature. After stirring at ambient temperature for 3 days, the reaction mixture is cooled to approximately 10° C. and the precipitate is filtered off. It is rinsed on the filter with 300 ml of dichloromethane. After drying, 105 g of a yellow powder are obtained, which powder is a 2/1 mixture of 1-hydroxy-5-phenyl-1,3-dihydroimidazole-2-thione and 1-hydroxy-4-phenyl-1,3-dihydroimidazole-2-thione.

97.7 g of this mixture are dissolved at reflux in THF (2 liters). After dissolving, 500 ml of heptane are added. Cooling is carried out to 15° C. The crystals are filtered off. After filtering and drying, 27 g (27.6%) of 1-hydroxy-5-phenyl-1,3-dihydroimidazole-2-thione are obtained.

Melting point=191.0° C.

¹H NMR (d₆-DMSO, 300 MHz): δ (ppm)=7.20 (s, 1H, H4); 7.34 (t, J=7 Hz, 1H, Ph-H); 7.42 (t, J=7 Hz, 2H, Ph-H); 7.66 (d, J=7 Hz, 2H, Ph-H); 11.60 (bs, 1H, —OH or NH); 12.35 (bs, 1H, —OH or NH).

¹³C NMR (d₆-DMSO, 300 MHz): 109.5; 126.7; 127.8; 128.2; 128.5; 129.0; 158.3.

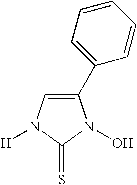

### Example 5

### Synthesis of 1-Hydroxy-4-phenyl-1,3-dihydroimidazole-2-thione

The mother liquors from the recrystallization of 1-hydroxy-5-phenyl-1,3-dihydroimidazole-2-thione obtained above in Example 4 are evaporated. 70 g of a mixture of 1-hydroxy-5-phenyl-1,3-dihydroimidazole-2-thione and 1-hydroxy-4-phenyl-1,3-dihydroimidazole-2-thione in the ratio 1/1 are obtained.

The 70 g of this mixture are recrystallized from a THF/toluene (0.7 l/1 l) mixture. After filtering and drying, 46.2 g of a mixture of 1-hydroxy-5-phenyl-1,3-dihydroimidazole-2-thione and 1-hydroxy-4-phenyl-1,3-dihydroimidazole-2-thione in the ratio 2/1 are obtained.

The mother liquors from this recrystallization are evaporated to dryness and the residue is triturated in methanol. 14.7 g of a mixture of 1-hydroxy-5-phenyl-1,3-dihydroimidazole-2-thione and 1-hydroxy-4-phenyl-1,3-dihydroimidazole-2-thione in the ratio 1/2 are thus obtained.

The 14.7 g of this mixture are recrystallized from 230 ml of methanol. After cooling, filtering and drying, 3.3 g (3.4%) of 1-hydroxy-4-phenyl-1,3-dihydroimidazole-2-thione are obtained.

Melting point=196.3° C.

¹H NMR (d₆-DMSO, 300 MHz): δ (ppm)=7.25 (t, J=7 Hz, 1 H, Ph-H); 7.44 (t, J=7 Hz, 2H, Ph-H); 7.73 (d, J=7 Hz, 2H, Ph-H); 7.86 (s, 1H, H-5); 11.76 (bs, 1H, —OH or NH); 12.70 (bs, 1H, —OH or NH).

¹³C NMR (d₆-DMSO, 300 MHz): 114.2; 124.2; 124.4; 127.9; 128.3; 129.2; 157.8.

MS (electrospray): m/z=381 (2M−H)⁻; 191 (M−H)⁻: m/z=193 (M+H)⁺.

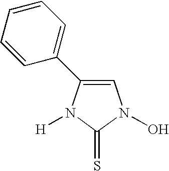

### Example 6

### Synthesis of 1-Hydroxy-5-(4-methoxyphenyl)-1,3-dihydroimidazole-2-thione:

### a) Preparation of 4-Methoxyphenylglyoxal

18.05 g (162 mmol) of selenium dioxide and 9 g of Fontainebleau sand are suspended in 65 ml of tetrahydrofuran and 20 ml of water. The mixture is brought to 40° C. until the reactant has dissolved.

25 g (166 mmol) of 4-methoxyacetophenone are added. The mixture is brought to reflux for approximately 70 h. The mixture is filtered through celite. The celite is rinsed with ethyl acetate and the filtrate is evaporated. The product obtained is recrystallized from water. After filtering and drying, 8.07 g of 4-methoxyphenylglyoxal (26.7%) are obtained.

### b) Preparation of 3-Hydroxy-4(5)-(4-methoxyphenyl)imidazole N-oxide

A solution of 6.11 g (88 mmol) of hydroxylamine hydrochloride in 7.5 ml of water is added dropwise, from 5 and 10° C., to a solution of 8 g (44 mmol) of 4-methoxyphenylglyoxal in 20 ml of methanol and 4.91 ml (1.5 eq.) of a 37% aqueous formaldehyde solution. At the end of the addition, 0.86 ml of concentrated hydrochloric acid is subsequently added dropwise and then the reaction mixture is allowed to return to ambient temperature. At the end of 24 h, a concentrated sodium hydroxide solution is added at 5° C. After stirring under cold conditions for 30 min, the precipitate formed is filtered off and rinsed with 20 ml of cold water. After drying under vacuum, 6.45 g (71.3%) of a 3-hydroxy-4(5)-(4-methoxyphenyl)imidazole N-oxide mixture are obtained.

### c) Synthesis of 1-Hydroxy-5-(4-methoxyphenyl)-1,3-dihydroimidazole-2-thione

Starting from 6.39 g (31 mmol) of 3-hydroxy-4(5)-(4-methoxyphenyl)imidazole N-oxide in 135 ml of methanol, 4.56 g of solid are obtained analogously to Example 4b and are recrystallized from a tetrahydrofuran/heptane (4/1) mixture. After filtering and drying, 2.24 g (32%) of 1-hydroxy-5-(4-methoxyphenyl)-1,3-dihydroimidazole-2-thione are obtained.

Melting point=150° C.

¹H NMR (d₆-DMSO, 400 MHz): δ (ppm)=3.77 (s, 3H); 6.99 (d, J=12 Hz, 2H, ArH); 7.07 (s, 1H, H4); 7.5 (d, J=12 Hz, 2H, ArH); 11.5 (s, 1H, —OH or NH); 12.3 (s, 1H, —OH or NH).

¹³C NMR (d₆-DMSO, 100 MHz): δ (ppm)=55.4; 108.2; 114.3; 120.2; 128.2; 128.3; 157.7; 159.2.

MS (electrospray): m/z=223 (M+H)⁺.

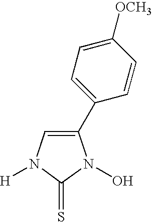

### Example 7

### Synthesis of 1-Hydroxy-4-(4-methoxyphenyl)-1,3-dihydroimidazole-2-thione

The mother liquors from the recrystallization of 1-hydroxy-5-(4-methoxyphenyl)-1,3-dihydroimidazole-2-thione obtained above in Example 6 are evaporated. The solid obtained is recrystallized several times from methanol. 0.719 g (10%) of 1-hydroxy-4-(4-methoxyphenyl)-1,3-dihydroimidazole-2-thione is obtained.

Melting point=160° C.

¹H NMR (d₆-DMSO, 400 MHz): δ (ppm)=3.77 (s, 3H); 6.9 (d, J=12 Hz, 2H, ArH); 7.6 (m, 3H, ArH+H5); 11.61 (s, 1H, —NH or —OH); 12.48 (s, 1H, —OH or NH).

¹³C NMR (d₆-DMSO, 100 MHz): δ (ppm)=55.4; 112.7; 114.5; 120.8; 124.4; 125.6; 157.2; 159.0.

MS (electrospray): m/z=223 (M+H)⁺.

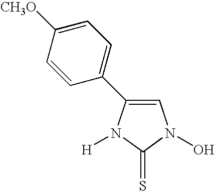

### Example 8

### Synthesis of 1-Hydroxy-5-(thiophen-2-yl)-1,3-dihydroimidazole-2-thione

### a) Preparation of 2-Thiophenylglyoxal

The desired compound is prepared in one stage from 50.0 g (396 mmol) of 2-acetylthiophene and 52.8 g (477 mmol) of selenium dioxide in 250 ml of tetrahydrofuran according to the process described [F. Kipnis, J. Ornfelt, J. Am. Chem. Soc., 68, 2734 (1946)]. After treating and recrystallizing from toluene, 24.23 g (44%) of 2-thiophenylglyoxal are obtained.

### b) Preparation of 3-Hydroxy-4(5)-(thiophen-2-yl)imidazole N-oxide

24 g (150 mmol) of 2-thiophenylglyoxal and 13.7 ml (0.184 mol) of a 37% aqueous formaldehyde solution are dissolved in 66 ml of methanol. A solution of 21.13 g (300 mmol) of hydroxylamine hydrochloride in 25 ml of water is added dropwise at 5-10° C. At the end of the addition, the appearance of a white solid is observed. 3 ml of concentrated hydrochloric acid are subsequently added dropwise and then the reaction mixture is allowed to return to ambient temperature. After stirring for 3 days, a solution of 2.16 g of hydroxylamine hydrochloride in 10 ml of water is added and the reaction mixture is heated at 50° C. for 2 h. The reaction mixture is allowed to return to ambient temperature. After filtering and drying under vacuum, 9.79 g (35%) of a 3-hydroxy-4(5)-(thiophen-2-yl)imidazole N-oxide mixture are obtained.

Melting point=182.9°-184.0° C.

### c) Synthesis of 1-Hydroxy-5-(thiophen-2-yl)-1,3-dihydroimidazole-2-thione

The desired compound is prepared analogously to Example 4b from 7.8 g (43 mmol) of 3-hydroxy-4(5)-(thiophen-2-yl)imidazole N-oxide. 1.13 g (13%) of 1-hydroxy-5-(thiophen-2-yl)-1,3-dihydroimidazole-2-thione are obtained.

Melting point=183.6°-184.8° C.

¹H NMR (d₆-DMSO, 300 MHz): δ (ppm)=7.11 (dd, J=5 Hz, J=3.6 Hz, 1H, H-4′); 7.25 (d, J=3 Hz, 1H, H-4); 7.43 (dd, J=3.6 Hz, J=1.2 Hz, 1H, H-3′); 7.55 (dd, J=5 Hz, J=1.2 Hz, 1H, H-5′).

¹³C NMR (d₆-DMSO, 75.5 MHz): δ (ppm)=107.9; 123.5; 124.9; 125.9; 127.3; 127.8; 157.5.

MS (electrospray): m/z=419 (2M+Na)⁺, 199 (M+H)⁺.

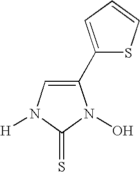

### Example 9

### Tyrosinase Activity Inhibition Assay

The activity of the inhibitors is measured starting from a lysate of B16F1 cells (murine melanoma line). In the presence of the L-tyrosine substrate, the tyrosinase present in these cells catalyses the hydroxylation of L-tyrosine to give L-DOPA and then the oxidation of the L-DOPA to give dopaquinone. In the presence of MBTH (3-methyl-2-benzothiazolinone hydrazone), the dopaquinone is trapped so as to form a pink complex which absorbs at 520 nm.

The B16F1 cells are cultured in DMEM medium+10% foetal calf serum+10⁻⁹ M α-MSH for 4 days at 37° C. under 7% CO₂. They are treated with trypsin, washed with PBS, counted and pelleted. The pellet is taken up at 10⁷ cells/ml in lysis buffer (10 mM sodium phosphate, pH 6.8−1% Igepal) and the suspension is treated with ultrasound for 10 seconds. After centrifugation for 30 minutes at 4000 rpm, the supernatant obtained constitutes the cell lysate used as tyrosinase source in the enzymatic assay.

The assays are carried out in duplicate in 384-well plates in a total volume of 50 μl. Each well contains:
40 μl of solution containing 1.25 mM L-tyrosine, 6.25 μM L-DOPA (cofactor) and 3.75 mM MBTH in buffer B (62.25 mM sodium phosphate, pH 6.8-2.5% dimethylformamide),
5 μl of inhibitor diluted in DMSO,
5 μl of cell lysate diluted to ½ in 50 mM Tris HCl buffer, pH 7.5.

The plate is incubated at 37° C. and a spectrophotometric reading is carried out at 520 nm after incubating for 6 hours. In order to avoid any possible absorption of the products, the system employs corrected absorbance (absorbance at time 6 h—absorbance at time zero).

The inhibitors are assayed in terms of dose-response so as to calculate an IC₅₀ (dose which inhibits 50% of the enzymatic activity).

Several internal controls are added to each experiment:
control for 100% activity: the 5 μl of inhibitor are replaced with 5 μl of DMSO,
control for 50% activity: the 5 μl of inhibitor are replaced with 5 μl of phenylthiourea at 300 μM in DMSO,
control for 0% activity: the L-tyrosine substrate is replaced with buffer B.

| Name | Molecular Structure | IC₅₀ (μM) |
|---|---|---|
| Compound 3 | 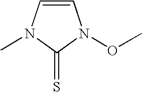 | 72.0 |
| Compound 4 | 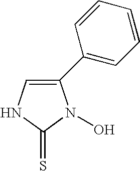 | 0.89 |
| Compound 6 | 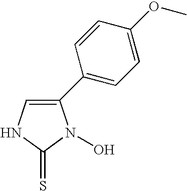 | 0.82 |
| Compound 8 | 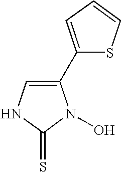 | 3.7 |

These results show that the compounds of formulae (I) and (II) have a good tyrosinase inhibitory activity.

### Example 10

### Formulation Examples

In this example, various specific formulations based on the compounds according to the invention have been illustrated.

Topically:

(a) Ointment:

| | | |
|---|---|---|
| Compound of Example 1 | 0.300 | g |
| White petrolatum, pharmaceutical grade | q.s. for 100 | g |

(b) Nonionic Water-In-Oil Cream:

| | | |
|---|---|---|
| Compound of Example 1 | 0.100 | g |
| Mixture of emulsive lanolin alcohols, | 39.900 | g |
| of waxes and of oils (“Anhydrous eucerin”, | | |
| marketed by BDF) | | |
| Methyl para-hydroxybenzoate | 0.075 | g |
| Propyl para-hydroxybenzoate | 0.075 | g |
| Sterile demineralized water | q.s. for 100 | g |

(c) Lotion:

| | |
|---|---|
| Compound of Example 8 | 0.100 g |
| Polyethylene glycol (PEG 400) | 69.900 g |
| 95% Ethanol | 30.000 g |

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for the treatment and/or prevention of a hyperpigmentation disorder, comprising administering to an individual, a thus effective amount of compound of the following formula (I): 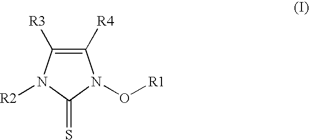
in which:
R1 is a hydrogen atom, an alkyl radical, an aralkyl radical or a cycloalkyl radical,
R2 is a hydrogen atom, an alkyl radical, an aryl radical or an aralkyl radical,
R3 is a hydrogen atom, an alkyl radical, an aryl radical or an aralkyl radical,
R4 is a hydrogen atom, an alkyl radical, an aryl radical or an aralkyl radical,
or a salt of the compounds of formula (I) or their optical, tautomeric and geometric isomers.

2. The regime or regimen as defined by claim 1, comprising administering a salt of a compound of formula (I) selected from among salts of alkali metals or alkaline earth metals or salts of zinc or of an organic amine.

3. The regime or regimen as defined by claim 1, said compound of formula (I) comprising at least one alkyl radical selected from among the methyl, ethyl, n-propyl, isopropyl, n-butyl, isobutyl, tert-butyl, n-pentyl and n-hexyl radicals.

4. The regime or regimen as defined by claim 1, said compound of formula (I) comprising at least one aryl radical selected from among the phenyl, biphenyl, naphthyl, thiophenyl, pyridinyl, pyrimidinyl, imidazolyl and triazolyl radicals, with the proviso that said aryl radicals may optionally be fused with one or more other rings and with the proviso that said aryl radicals may optionally be mono- or disubstituted by one or more substituents selected from among a halogen atom, a CF₃ radical, an alkyl radical, an alkoxy radical, a nitro functional group, an alkyl ester group, a nitrile functional group, an amide functional group, a carboxyl functional group, a hydroxyl radical and an amino functional group optionally substituted by at least one alkyl radical.

5. The regime or regimen as defined by claim 1, said compound of formula (I) comprising at least one aralkyl radical selected from among the radicals of formula —(CH₂)ₙ-aryl wherein n ranges from 1 to 6 inclusive and at least one aryl radical selected from among the phenyl, biphenyl, naphthyl, thiophenyl, pyridinyl, pyrimidinyl, imidazolyl and triazolyl radicals, with the proviso that said aryl radical may optionally be fused with one or more other rings and with the proviso that said aryl radical may optionally be mono- or disubstituted by one or more substituents selected from a halogen atom, a CF₃ radical, an alkyl radical, an alkoxy radical, a nitro functional group, an alkyl ester group, a nitrile functional group, an amide functional group, a carboxyl functional group, a hydroxyl radical and an amino functional group optionally substituted by at least one alkyl radical.

6. The regime or regimen as defined by claim 1, said compound of formula (I) comprising at least one alkoxy radical selected from among the methoxy, ethoxy, n-propoxy, isopropoxy, n-butoxy, isobutoxy, tert-butoxy, n-pentyloxy or n-hexyloxy radicals.

7. The regime or regimen as defined by claim 1, said compound of formula (I) comprising at least one halogen atom selected from among a chlorine, fluorine, bromine and iodine atom.

8. The regime or regimen as defined by claim 1, said compound of formula (I) comprising at least one cycloalkyl radical selected from among saturated cyclic hydrocarbon chains having from 3 to 8 carbon atoms.

9. The regime or regimen as defined by claim 1, said compound of formula (I) being selected from the group consisting of:
1-benzyloxy-1,3-dihydroimidazole-2-thione,
3-hydroxy-1,4-diphenyl-1,3-dihydroimidazole-2-thione,
3-hydroxy-1-(4-methoxyphenyl)-4-phenyl-1,3-dihydroimidazole-2-thione,
1-hydroxy-4-methyl-3,5-diphenyl-1,3-dihydroimidazole-2-thione, and
1-benzyloxy-3-methyl-1,3-dihydroimidazole-2-thione

10. The regime or regimen as defined by claim 1, comprising the treatment of a hyperpigmentary disorder selected from among melasma, chloasma, lentigines, senile lentigo, vitiligo, freckles, a post-inflammatory hyperpigmentation due to an abrasion and/or a burn and/or a scar and/or a dermatosis and/or a contact allergy; nevi, genetically determined hyperpigmentation, hyperpigmentation of metabolic or drug origin and a melanoma.

11. A compound having the following formula (II): 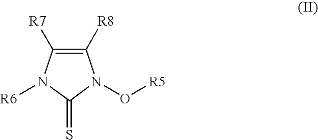
in which:
R5 is a hydrogen atom, an alkyl radical, an aralkyl radical or a cycloalkyl radical,
R6 is a hydrogen atom, an alkyl radical, an aryl radical or an aralkyl radical,
R7 is a hydrogen atom, an alkyl radical, an aryl radical or an aralkyl radical, and
R8 is a hydrogen atom, an alkyl radical, an aryl radical or an aralkyl radical

12. A compound selected from the group consisting of:
1-methoxy-1,3-dihydroimidazole-2-thione
1-(pyridin-3-ylmethoxy)-1,3-dihydroimidazole-2-thione
1-methoxy-3-methyl-1,3-dihydroimidazole-2-thione
1-hydroxy-5-(4-methoxyphenyl)-1,3-dihydroimidazole-2-thione
1-hydroxy-4-(4-methoxyphenyl)-1,3-dihydroimidazole-2-thione
1-hydroxy-5-(thiophen-2-yl)-1,3-dihydroimidazole-2-thione
1-benzyloxy-4-phenyl-1,3-dihydroimidazole-2-thione
1-benzyloxy-5-phenyl-1,3-dihydroimidazole-2-thione
1-ethoxy-4-phenyl-1,3-dihydroimidazole-2-thione
1-ethoxy-5-phenyl-1,3-dihydroimidazole-2-thione
1-ethoxy-4-methyl-1,3-dihydroimidazole-2-thione
1-ethoxy-5-methyl-1,3-dihydroimidazole-2-thione
1-ethyl-3-methoxy-1,3-dihydroimidazole-2-thione
1-benzyloxy-3-ethyl-1,3-dihydroimidazole-2-thione
1-(3,5-difluorobenzyloxy)-1,3-dihydroimidazole-2-thione
1-hydroxy-4-(3-nitrophenyl)-1,3-dihydroimidazole-2-thione
1-hydroxy-5-(3-nitrophenyl)-1,3-dihydroimidazole-2-thione
1-hydroxy-5-(4-nitrophenyl)-1,3-dihydroimidazole-2-thione
1-hydroxy-3-methyl-5-phenyl-1,3-dihydroimidazole-2-thione
1-hydroxy-3-methyl-4-phenyl-1,3-dihydroimidazole-2-thione
1-methoxy-4-phenyl-1,3-dihydroimidazole-2-thione
1-methoxy-5-phenyl-1,3-dihydroimidazole-2-thione

13. A medicament comprising a compound (II) as defined by claim 11.

14. A process for the preparation of a compound of formula (II) as defined by claim 11: 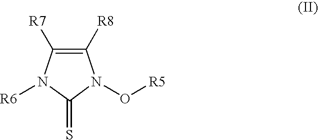
in which:
R5 and R6 are each a hydrogen atom,
R7 and R8, which are identical or different, are each a hydrogen atom, an alkyl radical, an aryl radical or an aralkyl radical,
which comprises the following successive stages:
a) addition of an aqueous formaldehyde solution to a compound of general formula (III): 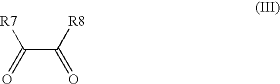
in solution in a polar solvent,
b) addition of an aqueous hydroxylamine hydrochloride solution to the mixture obtained in a),
c) addition of a hydrochloric acid solution to the mixture obtained in b),
d) neutralization and production of a precipitate,
e) filtering off and drying the precipitate obtained in d), corresponding to the compounds of general formulae (IVa) and/or (IVb): 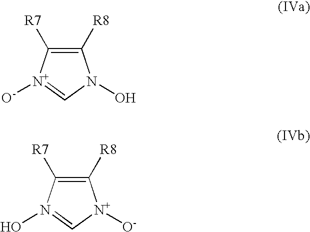
f) treatment of the compounds obtained in e) with a sulfur donor in order to obtain the compounds of general formula (II).

15. A process for the preparation of a compound of formula (II) as defined by claim 11: 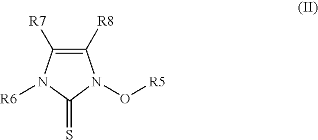
in which:
R5 is an alkyl radical, an aralkyl radical or a cycloalkyl radical and R6 is a hydrogen atom,
R7 and R8, which are identical or different, are each a hydrogen atom, an alkyl radical, an aryl radical or an aralkyl radical,
which comprises the following successive stages:
a) addition of an aqueous formaldehyde solution to a compound of general formula (III): 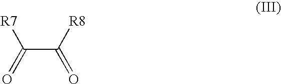
in solution in a polar solvent,
b) addition of an aqueous hydroxylamine hydrochloride solution to the mixture obtained in a),
c) addition of a hydrochloric acid solution to the mixture obtained in b),
d) neutralization and production of a precipitate,
e) filtering off and drying the precipitate obtained in d), corresponding to the compounds of general formulae (IVa) and/or (IVb): 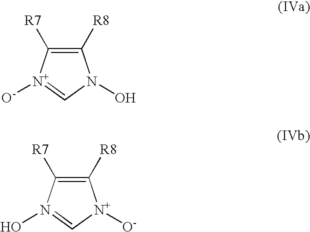
f) alkylation of the compounds obtained in e) to obtain the compounds of general formula (Va) and/or (Vb): 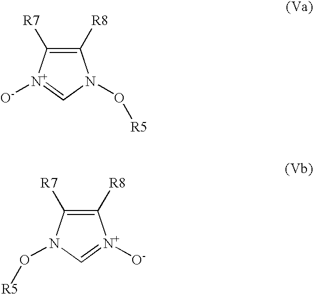
g) reduction of the compounds of general formula (Va) and/or (Vb) to obtain the compounds of general formula (VIa) and/or (VIb): 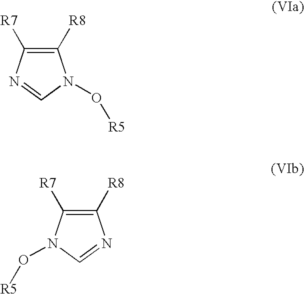
h) treatment of the compounds of general formulae (VIa) and/or (VIb) with n-butyllithium in an organic solvent and then with sulfur S₈, to obtain the compounds of general formula (II).

16. A process for the preparation of a compound of formula (II) as defined by claim 11: 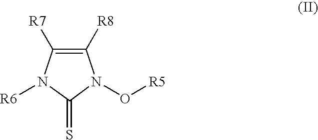
in which:
R5 is an alkyl radical, an aralkyl radical or a cycloalkyl radical and R6 is an alkyl radical or an aralkyl radical,
R7 and R8, which are identical or different, are each a hydrogen atom, an alkyl radical, an aryl radical or an aralkyl radical,
which comprises the following successive stages:
a) addition of an aqueous formaldehyde solution to a compound of general formula (III): 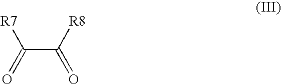
in solution in a polar solvent,
b) addition of an aqueous hydroxylamine hydrochloride solution to the mixture obtained in a),
c) addition of a hydrochloric acid solution to the mixture obtained in b),
d) neutralization and production of a precipitate,
e) filtering off and drying the precipitate obtained in d), corresponding to the compounds of general formulae (IVa) and/or (IVb): 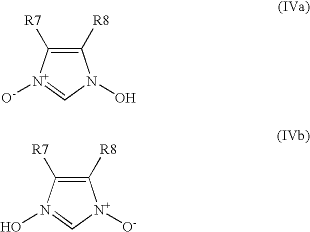
f) alkylation of the compounds obtained in e) to obtain the compounds of general formula (Va) and/or (Vb): 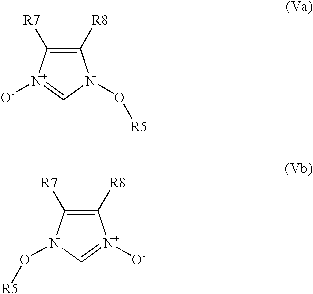
g) reduction of the compounds of general formula (Va) and/or (Vb) to obtain the compounds of general formula (VIa) and/or (VIb): 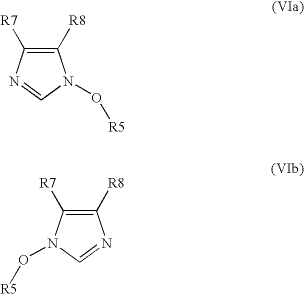
h) alkylation of the compounds obtained in g),
i) addition of a base and of sulfur S₈ to the reaction medium obtained in h), to obtain the compounds of general formula (II).

17. A pharmaceutical composition useful for the treatment of melasma, chloasma, lentigines, senile lentigo, vitiligo, freckles, a post-inflammatory hyperpigmentation due to an abrasion and/or a burn and/or a scar and/or a dermatosis and/or a contact allergy, nevi, genetically determined hyperpigmentation, hyperpigmentation of metabolic or drug origin or a melanoma, comprising a thus effective amount of a compound as defined by claim 11, formulated into a pharmaceutically acceptable vehicle therefor.

18. A pharmaceutical composition comprising at least one compound corresponding to the following formula (II): 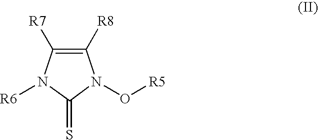
in which:
R5 is a hydrogen atom, an alkyl radical, an aralkyl radical or a cycloalkyl radical,
R6 is a hydrogen atom, an alkyl radical, an aryl radical or an aralkyl radical,
R7 is a hydrogen atom, an alkyl radical, an aryl radical or an aralkyl radical,
R8 is a hydrogen atom, an alkyl radical, an aryl radical or an aralkyl radical,
with the provisos that:
when R5 is a hydrogen atom, then R6 cannot represent a substituted or unsubstituted phenyl radical;
when R6 is a hydrogen atom or a methyl and R7 and R8 are each a hydrogen atom, then R5 cannot represent an unsubstituted benzyl radical;
when R5 and R6 are each a hydrogen atom and R7 is a phenyl radical, then R8 cannot represent a methyl,
or a salt or optical, tautomeric or geometric isomer thereof, formulated into a pharmaceutically acceptable vehicle therefor.

19. The pharmaceutical composition as defined by claim 18, comprising at least one salt of the compounds of formula (II) selected from among salts of alkali metals or alkaline earth metals or salts of zinc or of an organic amine.

20. The pharmaceutical composition as defined by claim 18, said compound of formula (II) comprising at least one alkyl radical having from 1 to 6 carbon atoms selected from among the methyl, ethyl, n-propyl, isopropyl, n-butyl, isobutyl, tert-butyl, n-pentyl and n-hexyl radicals.

21. The pharmaceutical composition as defined by claim 18, said compound of formula (II) comprising at least one aryl radical selected from among the phenyl, biphenyl, naphthyl, thiophenyl, pyridinyl, pyrimidinyl, imidazolyl and triazolyl radicals, with the proviso that said aryl radicals may optionally be fused with one or more other rings and with the proviso that said aryl radical may optionally be mono- or disubstituted by one or more substituents selected from among a halogen atom, a CF₃ radical, an alkyl radical, an alkoxy radical, a nitro functional group, an alkyl ester group, a nitrile functional group, an amide functional group, a carboxyl functional group, a hydroxyl radical and an amino functional group optionally substituted by at least one alkyl radical.

22. The pharmaceutical composition as defined by claim 18, said compound of formula (II) comprising at least one aralkyl radical selected from among the radicals of formula —(CH₂)ₙ-aryl wherein n ranges from 1 to 6 inclusive and at least one aryl radical selected from among the phenyl, biphenyl, naphthyl, thiophenyl, pyridinyl, pyrimidinyl, imidazolyl and triazolyl radicals, with the proviso that said aryl radical may optionally be fused with one or more other rings and with the proviso that said aryl radical may optionally be mono- or disubstituted by one or more substituents selected from among a halogen atom, a CF₃ radical, an alkyl radical, an alkoxy radical, a nitro functional group, an alkyl ester group, a nitrile functional group, an amide functional group, a carboxyl functional group, a hydroxyl radical and an amino functional group optionally substituted by at least one alkyl radical.

23. The pharmaceutical composition as defined by claim 18, said compound of formula (II) comprising at least one alkoxy radical selected from among the methoxy, ethoxy, n-propoxy, isopropoxy, n-butoxy, isobutoxy, tert-butoxy, n-pentyloxy or n-hexyloxy radicals.

24. The pharmaceutical composition as defined by claim 18, said compound of formula (II) comprising at least one halogen atom selected from among a chlorine, fluorine, bromine and iodine atom.

25. The pharmaceutical composition as defined by claim 18, said compound of formula (II) comprising at least one cycloalkyl radical selected from among saturated cyclic hydrocarbon chains having from 3 to 8 carbon atoms.

26. The pharmaceutical composition as defined by claim 18, said compounds of formula (II) are those for which at least one and optionally all of the following conditions are observed:
R5 is a hydrogen atom, an alkyl radical or an aralkyl radical,
R6 is a hydrogen atom, an alkyl radical or an aryl radical,
R7 is a hydrogen atom, an alkyl radical or an aryl radical,
R8 is a hydrogen atom, an alkyl radical or an aryl radical,
and their salts and their optical, tautomeric and geometric isomers.

27. The pharmaceutical composition as defined by claim 18, said compounds of formula (II) are those for which at least one and optionally all of the following conditions are observed:
R5 is a hydrogen atom,
R6 is a hydrogen atom,
R7 is a hydrogen atom, an alkyl radical or an aryl radical,
R8 is a hydrogen atom or an aryl radical,
and their salts and their optical, tautomeric and geometric isomers.

28. The pharmaceutical composition as defined by claim 18, wherein the concentration of compound(s) of formula (II) ranges from 0.001% to 10% by weight, with respect to the total weight of the composition.

29. The pharmaceutical composition as defined by claim 18, wherein the concentration of compound(s) of formula (II) ranges from 0.01% to 1% by weight, with respect to the total weight of the composition.

30. A cosmetic composition comprising at least one compound as defined by claim 18, formulated into a cosmetically acceptable vehicle therefor.

31. The cosmetic composition as defined by claim 30, wherein the concentration of compound(s) ranges from 0.001% to 3% by weight, with respect to the total weight of the composition.

32. A regime or regimen for preventing and/or treating signs of aging and/or dry skin, comprising administering to an individual, a thus effective amount of a cosmetic composition as defined by claim 30.
